(19) United States
(12) Patent Application Publication
(10) Pub. No.: US 2010/0312073 A1
(43) Pub. Date: Dec. 9, 2010

(54) METHOD OF PREDICTING PAIN MEDICATION EFFICACY

(76) Inventor: David Yarnitsky, Haifa (IL)

Correspondence Address: YORAM TSIVION, P.O. BOX 1307, PARDES HANNA, 37111, IL

(21) Appl. No.: 12/864,881

(22) PCT Filed: Jan. 29, 2009
(86) PCT No.: PCT/IB09/50366

§ 371 Date: Jul. 28, 2010

Related U.S. Application Data
(60) Provisional application No. 61/024,925, filed on Jan. 31, 2008.

(51) Int. Cl. A61B 5/00

(52) U.S. Cl. 600/300

(57) ABSTRACT

A predictive method that aims at matching a specific pain medication, particularly of the SNRI type, to a specific patient is disclosed. In accordance with the predictive method, DNIC pain modulation is first performed on the patient who is about to receive a pain medication treatment. The results of the test are then taken as is or optionally transformed to construct a value. By analyzing this value, a specific pain medication can be matched to the aforementioned patient.

### CROSS-REFERENCE TO RELATED APPLICATIONS

The present application claims the benefit of priority to U.S. Provisional Patent Application Ser. No. 61/024,925, filed Jan. 31, 2008, entitled “METHOD OF PREDICTING PAIN MEDICATION EFFICACY;” the aforementioned application is incorporated herein by reference.

### FIELD OF THE INVENTION

The present invention relates to diagnostic methods in general. More particularly, the present invention relates to a method of predicting the efficacy of particular type of pain medications in a forthcoming treatment of a given subject.

### BACKGROUND OF THE INVENTION

Pain sensation is the result of peripherally generated neural stimuli which are transmitted therefrom and modulated in the CNS before its arrival in the cortex, and in the consciousness. Thus, the same external stimulus will evoke different perceptions among different people, pending on their modulation processes.

Modulation mechanisms tests known in the art include: 1—temporal summation (TS) and 2—diffuse noxious inhibitory control (DNIC). The mechanism underlying TS is excessive activation of N-methyl-D-aspartate (NMDA) receptors in response to high levels of nociceptive input, clinically manifested by allodynia and hyperalgesia. TS is tested by application of repetitive stimuli, with concomitant assessment of the increase in pain scores over time. Diffuse noxious inhibitory control (DNIC) represents the endogenous analgesia system, where modulatory effect is exerted on incoming spinal nociceptive data. This phenomenon is based on a spinal-bulbar-spinal loop, under cerebral control, which, at least partially, is opioid-mediated. There is a growing body of evidences from animal studies pointing to the important role of spinal serotonin (5-HT) and noradrenaline (NA) in mediation of pain inhibition via DNIC. DNIC is typically tested in the lab using the ‘pain inhibits pain’ paradigm, by two remote noxious stimuli with one, the ‘conditioning’ pain, inhibiting the other, the ‘test’ pain.

In recent years many reports disclose the involvement of both TS and DNIC in altered pain modulation in chronic pain patients. Enhanced TS was found in chronic idiopathic pain patients such as TMD (temporomandibular disorder) (Maixner et al., 1998), fibromyalgia (Graven-Nielsen et al., 2000), low back pain (George et al., 2006), tension headache and musculoskeletal pain (Kleinbohl et al., 1999). Similarly, less-efficient DNIC response was found in many of the syndromes such as TMD (Maixner et al., 1995), fibromyalgia (Lautenbacher and Rollman), tension headache (Pielsticker et al., 2005) and irritable bowel syndrome (IBS) (Chang, 2005).

Altered pain modulation profile with decreased endogenous analgesia as shown by less efficient DNIC and enhanced central sensitization (increased temporal summation), was exhibited by patients with several idiopathic pain syndromes, as described elsewhere.

Noticeably, the modulation in the various pain syndromes mentioned above, were performed on patients already suffering from chronic pain. It is as yet unclear whether the alterations in pain modulation are (1) a consequence of the long experience of pain, reflecting sensitization of pain pathways expressed by increased TS and/or by decreased DNIC efficiency or (2) a cause of the clinical pain syndrome, where pre-existing modulation properties such as increased summation and less efficient DNIC expose the subject to acquire the pain syndrome.

Chronic post-operative pain (CPOP) is now considered a disease on its own merit and is often resistant to therapy. It is associated with decreased health-related quality of life, limits daily activities and causes psychosocial distress (Hatter et al., 2000). Thoracotomy causes one of the highest relative incidences of CPOP, with chronic post-thoracotomy pain (CPTP) occurring in 15-60% of patients (Perttunen et al., 1999). CPTP is considered, in most cases, as neuropathic pain because of its pain characteristics, sensory loss, allodynia and hyperalgesia. Further, intercostal nerve injury during surgery is almost unavoidable (Benedetti et al., 1997). Since a portion of patients suffer CPTP (despite similar surgical procedures), and since the degree of intra-costal nerve lesion has been reported not be associated with chronic pain intensity or altered cutaneous sensation (Maguire et al., 2006b), another systemic factor is likely to be involved, namely, altered pain modulation profile (Kehlet et al., 2006).

Acute post-operative pain is one of the most pertinent factors in the generation of CPOP (Katz et al., 1996). Two parameters of the acute post-operative pain have been proposed as predictors of chronic pain—pain magnitude in the immediate post-operative stage, and the neuroplastic changes evoked after surgery around the scar (allodynia/hyperalgesia). Both may reflect the balance between descending facilitation and deficient inhibitory mechanisms (Kehlet at al., 2006). Due to the importance of acute post-operative pain, several prospective studies were conducted applying static quantitative sensory testing (QST). Some of them showed that acute post-operative pain can be predicted by preoperative pain assessment of pain thresholds or supra-threshold magnitude estimations (Bisgaard et al., 2001).

The one prospective study that tried to predict CPOP, used static psychophysical measures in response to cold pain stimulation failed to identify patients at risk (Bisgaard et al., 2005). To date, no predictive studies for both acute and chronic post-operative pain, which are based on modulation profile obtained by dynamic pain psychophysics tests has been reported, let alone for determining the efficacy of neuropatic pain medication.

Therapy for neuropathic pain, despite newly presented drugs, is still frustrating, with less than half of the patients not achieving satisfactory relief; possibly due to the lack of mechanism-oriented choice of therapy. Currently, it is mainly the side effects that lead the physician in choosing the anti-neuropathic pain medication, whereas ideally its mechanism of action should be the leading consideration. Several lines of pharmacological therapy are recommended for neuropathic pain; antidepressants, antiepileptics and opioids.

Antidepressants include tricyclics and SNRIs (Serotonin and noradrenaline reuptake Inhibitor), since SSRIs (selective serotonin reuptake inhibitors) have proven less effective in treating neuropathic pain. Tricyclics have been the mainstay of therapy for many years, giving a fairly good number need to treat (NNT) of 2-3, but with considerable adverse effects especially in older patients (Watson et al., 1998). SNRIs such as venlafaxine and duloxetine have proven as effective for neuropathic pain, mainly for diabetic neuropathy (Goldstein et al., 2005), with a slightly less favourable NNT (4-6), but more favourable side effect profile (Wernicke et al., 2007). The mechanism of action of both tricyclics and SNRIs is to increase synaptic levels of both Serotonin (5-hdroxytryptamine or 5-HT) and noradrenaline (NA), via a dual inhibition of their reuptake in the CNS. An increased level of these neurotransmitters exerts descending modulation via the bulbo-spinal tracts, augmenting the inhibitory effect on pain perception. Of the antiepileptics, the medications that seem to be most relevant for neuropathic pain are gabapentin (GBP) and pregabalin (PGB) (Chandra et al., 2006), whereas oxcarbamazepine and lamotrigine showed lesser effects (Viniket al., 2007). PGB and GBP inhibit the presynaptic α-2-δ subunit of the Ca channel, and are therefore expected to diminish effects that depend on calcium influx, including central sensitization. Between GBP and PGB, the latter is preferable due to more linear pharmacokinetics, and more predictable results, with a narrower window of effective dosages compared to GBP (Cruccu, 2007). The role of opioids in neuropathic pain is still controversial; while convincing evidence has been accumulated in recent years for efficacy in neuropathic pain, the safety profile is still unclear, leading the recent EFNS guideline to recommend opioids as second line medications for neuropathic pain (Attal et al., 2006).

Noticeably, as mentioned above, the mechanism targeted by the aforementioned SNRI type of pain medications is the same mechanism tested by DNIC, namely, increase of synaptic levels of both 5-HT and NA via a dual inhibition of their reuptake into the CNS, which as known in the art characterize altered modulation profile of idiopathic pain patients. Hence DNIC is not fruitful just for determining the predisposition of a subject to a pain condition but also can be beneficially employed to assess the susceptibility of a subject to a SNRI type of pain medications and/or to predict the efficacy of SNRI medications in a forthcoming treatment, due to the same mechanism underlying both DNIC testing and SNRI medications; whereby providing for educated and efficient preventive therapy in patients prone to develop chronic pain, such as those about to undergo specific surgeries or other interventions. This way, patients with abnormal mechanisms of pain modulation would benefit from prescription of an anti-neuropathic pain medication of a specific type whose pharmacological mechanism intervenes with their individual pain modulation mechanism.

### BRIEF DESCRIPTION OF THE DRAWINGS

The present invention will be understood and appreciated more fully from the following detailed description taken in conjunction with the appended drawings in which:

FIG. 1 is a chart describing the steps carried out in a procedure of a prediction method of the present invention;

FIG. 2 is a plot of empirical data demonstrating a correlation between DNIC efficiency and intensity of CPTP (6-12 months);

FIG. 3 is a plot of empirical data demonstrating a correlation between TS and CPTP (6-12 months);

FIG. 4 is a bar chart of empirical data demonstrating combined effects of DNIC and TS on CPTP intensity;

FIG. 5 is a bar chart of empirical data demonstrating the correlation between low base line of DNIC scores and an increased response to duloxetine vs. placebo treatment.

While the invention is susceptible to various modifications and alternative forms, specific embodiments thereof have been shown by way of example in the drawings. It should be understood, however, that the description herein of specific embodiments is not intended to limit the invention to the particular forms disclosed, but on the contrary, the intention is to cover all modifications, equivalents, and alternatives falling within the spirit and scope of the invention.

### DISCLOSURE OF THE INVENTION

Illustrative embodiments of the invention are described below. In the interest of clarity, not all features of an actual implementation are described in this specification. It will be appreciated that in the development of any such actual embodiment, numerous implementation-specific decisions must be made to achieve the developers' specific goals, such as compliance with system-related and business-related constraints, which vary from one implementation to another. Moreover, it will be appreciated that such a development effort might be complex and time-consuming, but would nevertheless be a routine undertaking for those of ordinary skill in the art having the benefit of this disclosure.

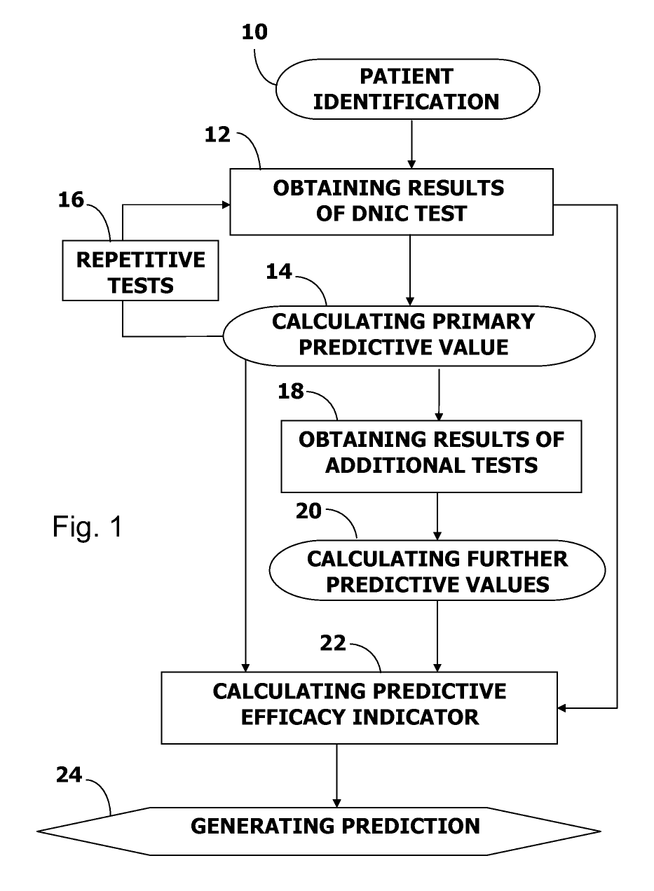

The predictive method of the invention aims at matching a specific pain medication, particularly of the SNRI type to a specific patient. In accordance with the invention, DNIC pain modulation is first performed on a patient who is about to receive a pain medication treatment. The results of the test are then be taken as is or optionally transformed to construct a value referred to hereinafter as primary predictive value. To describe the entire process for matching a medication to a patient, reference is made to FIG. 1, in which a schematic representation of an exemplary procedure of the prediction method of the present invention is shown. The patient is initially identified, at step 10, according to the criteria elaborated infra. The patient is then subjected to DNIC pain modulation testing, at step 12, and the scores of the testing are acquired; whereby a primary predictive value is obtained, at step 14.

Optionally, the patient is subsequently subjected to a plurality of DNIC tests, at step 16, in order to attain a statistical significance of the scores. Subsequent to that, the scores can be subjected to linear regression or alternative mathematically analysis procedures; whereby primary predictive value is calculated, at step 14.

In addition to the primary predictive value directly based on pain perception, namely DNIC, further predictive values based on pain perception, such as TS test scores, QST scores (pain thresholds and supra-threshold stimuli) and post-operative (PO) pain scores, as well as values not based on pain perception, for example personality, age, and gender, may be employed. All the predictive values can be used for the processing of the predictive efficacy indicator, as elaborated infra.

Accordingly, subsequent to that, the patient may optionally be subjected to additional non-DNIC testings, at step 18, and further predictive values can be consequently obtained and/or calculated, at step 20.

If there is a plurality of scores obtained from multiple DNIC or non-DNIC tests, the scores are preferably to be plotted and assessed with linear regression or alternatively mathematically processed to yield a predictive value. The linear equation parameters of the regression line can be the primary and further predictive values calculated, at step 20.

Then, the predictive efficacy indicator is obtained and/or calculated, at step 22, and typically compared to a predetermined value, whereby the susceptibility of a subject to a SNRI type of pain medications and/or the efficacy of SNRI medications in a forthcoming treatment assessed and a corresponding prediction is generated, at step 24.

If no repetitive DNIC tests are performed, the scores of the single DNIC test become the primary predictive valuse and the primary predictive value can be the only source of input data for determining the predicted efficacy indicator if no additional non-DNIC testings are performed; hence, accordingly, the scores of the single DNIC test can be the only source of input data for determining the predictive efficacy indicator.

These steps may be followed by a prescription of a particular, most suitable, pain medication for the given subject, according to the aforementioned prediction method.

According to an exemplary testing procedure, the DNIC can be assessed as the difference of pain ratings to ‘test stimulus’ applied before and during the immersion of the contralateral hand into hot water bath (conditioning stimulus) as elaborated infra. Exemplary test stimulus of a 30 seconds period with contact heat, such as 30×30 mm² thermode TSA-2001 available from Medoc, can be delivered for instance on the non-dominant forearm at the individually-predetermined temperature that evoked pain intensity of 60 on a 0-100 NPS (more details in Granot et al., 2006). The stimulus temperature can be raised by 2° C./sec from 32° C. to the destination temperature. Fifteen minutes after the completion the first pre-immersion test, the patient is asked to immerse his/her dominant hand for 1′ into the water bath of 46.5° C. (Neto Cooling Bath CBN 8-30, Allerod, Denemark), while second application of the ‘test stimulus’ is repeated during last 30 seconds of immersion. To assess the residual DNIC effect after end of the immersion, the ‘test stimulus’ may be applied for the third time. During each application of ‘test stimulus’ the patient rates his/her thermal pain intensity every 10 seconds, (more details in Granot et al., in press).

Exemplarily, mechanically evoked stimuli can be assessed by contacting a subject with the 6.45 mN von Frey filament (Stoeteling Ltd. US).

A non-limiting list of means for evoking and/or conditioning stimuli in manner include water baths, a heating surface, a soft surface on which a patient puts an hand, and is subjected to either heating or cooling, a plurality of small surfaces in contact with the hand each heating or cooling, an array of electrodes that give electrical stimuli, optionally in a few places on the hand, a blood pressure cuff to be inflated for a short time, causing pain, a thermal glove or a glove like made of two surfaces that can be opened in-between two heating elements, optionally including a few sources over the hand, combination of two or more modalities of stimulation out of thermal, electrical, mechanical, chemical, etc.

To assess TS, single stimulus and then a consequence of 10 successive stimuli can be applied for instance to the dominant volar forearm and bilaterally to the masseter muscle area. Pricking pain scores (0-100 on NPS) should be obtained after the single and then at the end of the successive stimuli. The difference between these scores will be calculated as TS.

The rating of the score tests is preferably carried out with reference to the numerical pain scale (NPS) as elaborated in the publications of Ayesh et al., 2007 and Baad-Hansen et al., 2003 and/or by brief pain inventory (BPI), as elaborated in the publication of Cleeland & Ryan, 1994; all the publications referenced above are incorporated herein by reference. A plurality of repetitive tests may be performed on a subject to attain a reliable statistic significance of the scores.

### APPLICATION OF THE INVENTION

The prediction method of the present invention can be beneficially performed on any individual who is expected to or experiences pains, particularly idiopathic and neuropathic pains, for instance due to a specific medical treatment that is known to be accompanied by such.

Additional pertinent criterion is an anticipated or actual prescription of tricyclics and/or SNRIs such as venlafaxine and duloxetine or any other type of pain medication that shares the same targeted mechanism of action, which is an increase of synaptic levels of both 5-HT and NA, via a dual inhibition of their reuptake in the CNS.

Thus the applications of the prediction method are mainly for idiopathic and neuropathic pains, for which tricyclics and/or SNRIs medications are primarily indicated. These include diabetic neuropathy, uremic neuropathy, post herpetic neuralgia, post operative neruopathic pain, low back pains, traumatic nerve lesions usually to the limbs, neuropathy due to chemotherapy which is known to be caused by vincristine, taxol, platinot, thalidomide and a few additional new chemo agents can cause pain neuropathy, hereditary neuropathy, pesticide induced neuropathy, and many other situations.

The applications of the prediction method to non-neuropathic pains include temporomandibular disorders, fibromyalgia, vulvar vestibulitis, irritable bowel, tension type headache, pain in the chronic fatigue syndrome and any other pain condition mentioned supra.

Additional application of the prediction method is for individuals who are likely to develop pain, such as post-operative pains.

### UTILITY OF THE INVENTION

### Example 1

In this study, data from 84 patients (mean age 61.5±13.7) who had undergone elective thoracotomy in the Department of Thoracic Surgery, at Rambam Medical Center, and met the study inclusion criteria were analyzed. All psychophysical tests were conducted the day before surgery when patients were pain free. Acute PO pain scores were recorded twice at the morning hours of the 2nd (with) and 5th (without epidural catheter for pain control) days after surgery in 3 conditions: rest, arm elevation at the surgery side and deliberate coughing.

The primary and additional predictive values, namely DNIC and TS respectively, were operationally defined in two ways: as a binary trait, e.g., whether or not there is at least 50% pain reduction, and as a semi-quantitative value assessing the degree (%) of pain reduction at the end of treatment. The first type of values was assessed with logistic regression modelling, while the second type was assessed with linear regression.

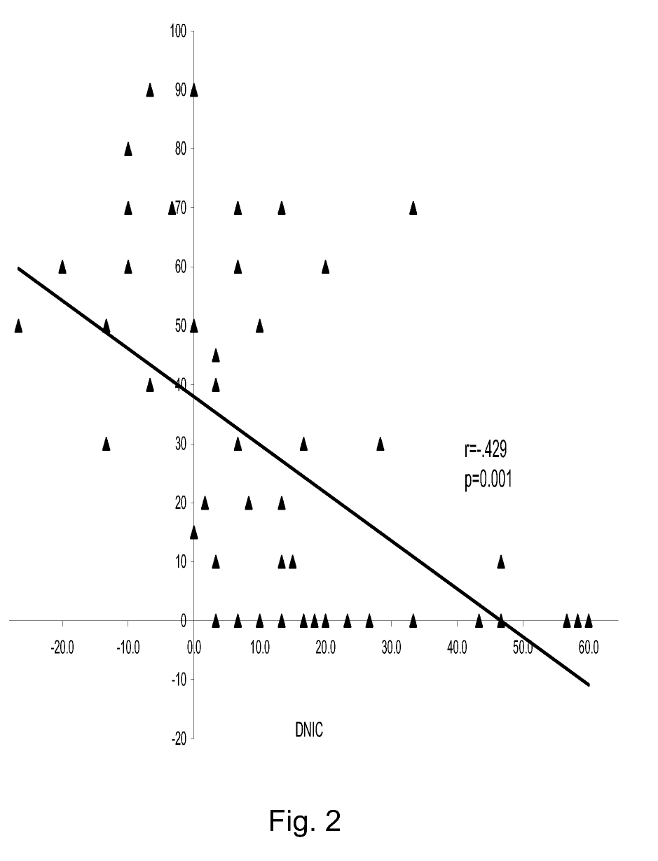

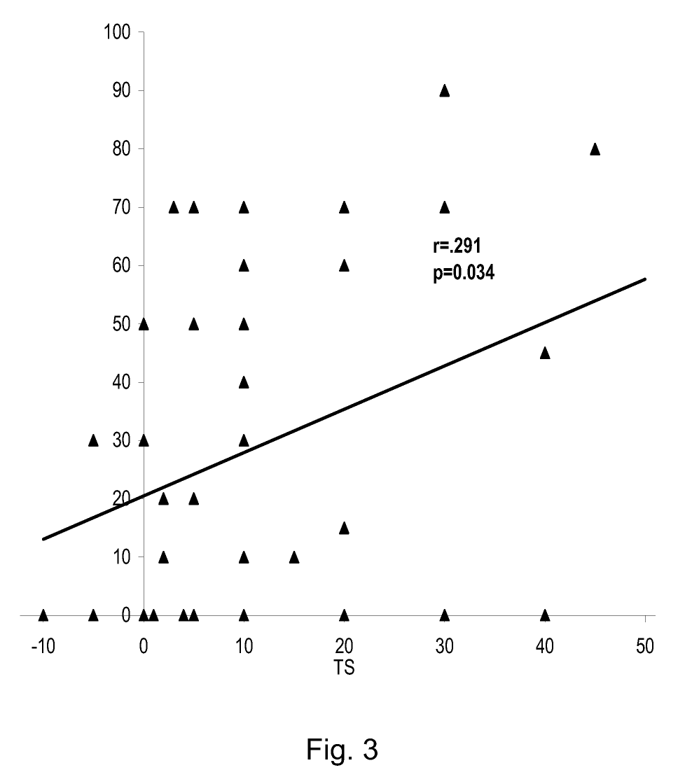

It was found that DNIC efficiency was negatively correlated with chronic pain (r=−0.429, p=0.001) but not with acute pain scores, as can be seen from the chart shown in FIG. 2 to which reference is now made. It also was found that TS extent correlated with chronic pain (r=0.295, p=0.039) as can be seen from the chart shown in FIG. 3 to which reference is now made. Logistic regression models revealed that DNIC predicts the risk for chronic post-thoracotomy pain, with OR of 0.50. Linear regression model (R²=0.247, p=0.001) shows that both DNIC (B=−0.678, t=−3.28, p=0.002) and TS (B=0.610, t=2.01, p=0.050) independently predict chronic post thoracotomy pain (CPTP) intensity. Moreover, CPTP patients were divided into 4 subgroups according to the combination of positive or negative DNIC with positive or negative TS.

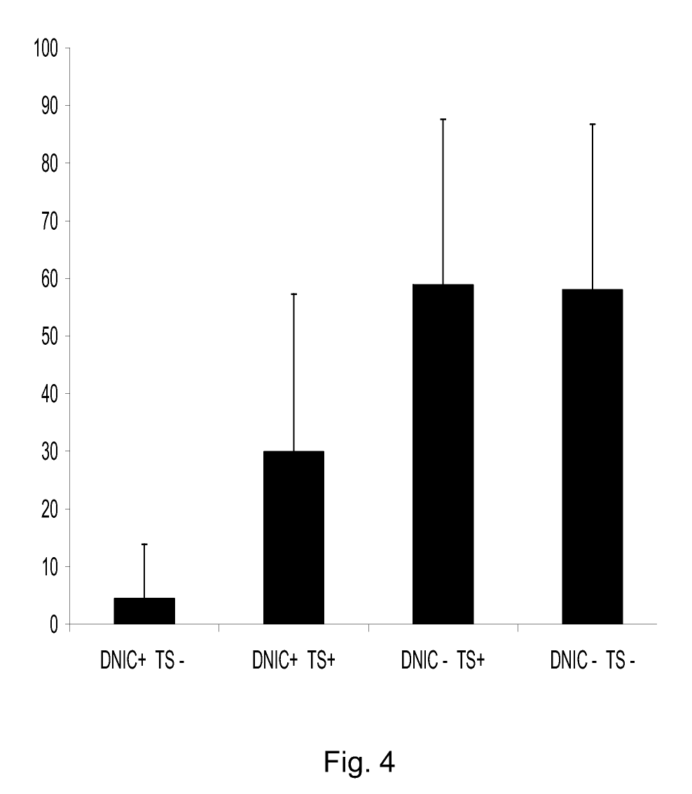

Significant relation was found between the sub-group of combined modulation profile and the presence of CPTP (Chi=22.1, P=0.001). Pain scores at the chronic stage for each sub-group are shown in FIG. 4, to which reference is now made, where DNIC+ represents increased DNIC efficiency (reduction of ‘test pain’ scores due to the ‘conditioning stimulus’), and TS+ represents enhanced pain summation, (increase in pain scores along series of repetitive stimuli). This demonstrate the dominant role of DNIC over TS in determining chances for chronic pain. Thus, the right two columns represent patients with non efficient DNIC, who have high chance for chronic pain regardless of their TS. For those with efficient DNIC, high TS did increase the chances for chronic pain (second column from left). No association was found between TS and DNIC themselves, suggesting that these two dynamic psychophysical tests represent two seemingly unrelated mechanisms of pain modulation. No association was found between chronic pain and the static QST measures (pain thresholds and scoring of supra-threshold stimuli for thermal and mechanical stimuli), age, education, gender and the pain related personality values of state and trait anxiety as well as pain enhancement.

### Example 2

In this study, a randomized double blind placebo controlled cross-over design was performed on 40 healthy volunteers aged 21-38 yrs, using Duloxetine 60 mg once a day for one week, and non active placebo, with one week washout in-between treatments (20 volunteers did not complete the second week due to administrative reasons). DNIC paradigm, tested at baseline and after each of the treatments, consisted of administration of two painful stimuli, a test-pain delivered by contact heat, and a ‘conditioning’ pain induced by hot water immersion of the other hand. The DNIC efficiency was calculated as the difference between pain perceptions induced by the test-pain when given alone, and when given concomitantly with the conditioning one.

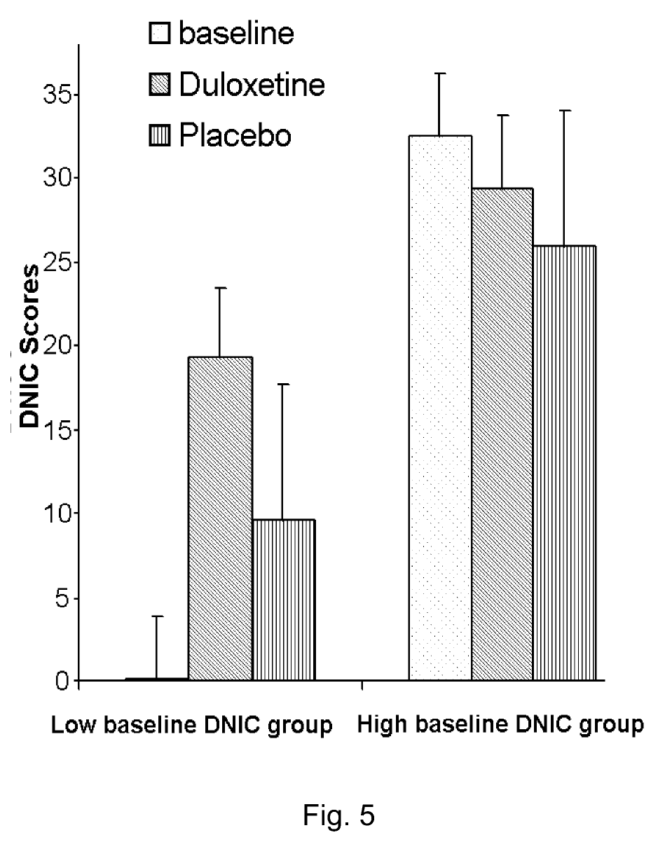

Mixed model ANOVA indicated a significant difference between treatment DNIC scores (P=0.0082) and pre-treatment DNIC scores (P=0.0896), for one weak treatment. Tukey tests indicated that Duloxetine treatment was only significantly effective for the low pre-treatment DNIC group (pre-treatment vs. Duloxetine treatment DNIC score, 0.15 vs. 19.35, P<0.05), and not for the high pre-treatment DNIC group (32.50 vs. 29.26, NS), with placebo ineffective for either group, as shown in FIG. 5, to which reference is now made.

The higher effect found in individuals with lower DNIC scores confirms that these individuals are more likely to benefit clinically from this type of pain medications. Less efficient DNIC is (i) associated with presence of idiopathic pain syndromes, and (ii) predicts development of chronic post operative pain. Measuring DNIC before use of Duloxetine and/or other SNRI type medications, in order to predict whether it will or will not be beneficial to the specific patient, is a considerable step towards individually tailored therapy in pain medicine.

It will be appreciated that the present invention is not limited by what has been particularly described and shown hereinabove and that numerous modifications, all of which fall within the scope of the present invention, exist. Rather the scope of the invention is defined by the claims which follow:

1. A method of predicting the efficacy of pain medications selected from the group consisting of SNRI and tricyclics, said method comprising the steps of:
identifying a subject;
subjecting said subject to at least a single DNIC test;
obtaining scores of said DNIC test, and
analyzing said scores;
wherein lower scores at said DNIC test are associated with a higher efficacy of said treatment, and
whereby said efficacy is predicted.

2. The method as in claim 1, further comprising the step of prescribing said subject with a pain medication.

3. The method as in claim 1, wherein said subject is expected to or undergoes a condition associated with pain.

4. The method as in claim 3, wherein said condition is selected from the group consisting of: idiopathic pain, neuropathic pain, diabetic neuropathy, uremic neuropathy, post herpetic neuralgia, post operative neruopathic pain, low back pains, traumatic nerve lesions, neuropathy due to chemotherapy, pain neuropathy, hereditary neuropathy, pesticide induced neuropathy; temporomandibular disorders, fibromyalgia, vulvar vestibulitis, irritable bowel, tension type headache, musculoskeletal pain, pain in the chronic fatigue syndrome, post-operative pain, chronic post-operative pain and any variation thereof.

5. A method as in claim 1, wherein a stimulus for said DNIC test is selected from the group consisting of: thermal, electrical, mechanical and chemical and any combination thereof.

6. A method as in claim 1, wherein a stimulus for said DNIC test is evoked by a means selected from the group consisting of: water bath, heating surface, a soft surface on which is contacted with said subject and it either heats or cools, a plurality of small surfaces in contact with said subject each heating or cooling, an array of electrodes that give electrical stimuli, a blood pressure cuff to be inflated for a short time, a thermal glove, combination and variation thereof.

7. A method as in claim 1, further comprising a step selected from the group consisting of:
subjecting said subject to repetitive DNIC tests;
subjecting said subject to additional non-DNIC testing;
calculating a primary predictive value;
calculating further predictive values, and
calculating a predictive efficacy indicator.

8. A method as in claim 7, wherein said additional non-DNIC testing is selected from the group consisting of: tests based on pain perception—TS test, QST tests, pain thresholds, supra-threshold stimuli, post-operative (PO) pain; tests that are not based on pain perception—personality, age, and gender.
